(19) United States
(12) Patent Application Publication
(10) Pub. No.: US 2018/0206613 A1
(43) Pub. Date: Jul. 26, 2018

(54) MAKEUP STORAGE AND APPLICATION SYSTEM

(71) Applicant: Margarita Sidorova, Etobicoke (CA)

(72) Inventor: Margarita Sidorova, Etobicoke (CA)

(21) Appl. No.: 15/413,986

(22) Filed: Jan. 24, 2017

(51) Int. Cl. A45D 40/24; A61K 8/02; A61K 8/73; A61K 8/20; A61K 8/19; A61K 8/26; A61K 8/97; A61K 8/64; A61K 8/29; A61K 8/92; A61K 8/891; A61K 8/34; A61K 8/86; A61K 8/36; A61K 8/60; A61K 8/67; A61K 8/27; A61K 8/365; A45D 40/00; A45D 40/26; A45D 33/18; B65D 51/24

(52) U.S. Cl. CPC A45D 40/24; A61K 2800/882; A61K 8/0216; A61K 8/732; A61K 8/20; A61K 8/19; A61K 8/26; A61K 8/97; A61K 8/64; A61K 8/29; A61K 8/922; A61K 8/891; A61K 8/927; A61K 8/925; A61K 8/342; A61K 8/86; A61K 8/361; A61K 8/60; A61K 8/678; A61K 8/975; A61K 8/27; A61K 8/34; A61K 8/365; A61K 8/36; A45D 40/0068; A45D 40/262; A45D 33/18; B65D 51/24; A61K 2800/43; A61K 2800/87; A61K 8/022

(57) ABSTRACT

A makeup storage and application system is disclosed herein. The makeup storage and application system includes a makeup storage and application assembly having a makeup palette for holding, a base powder, a wax, a cream, and at least one pigment. The makeup palette holds the base powder, the wax, the cream and at least one pigment. The at least one pigment is able to be interchangeably mixed from the makeup palette with the base powder, the wax, and the cream to create a customized makeup for a user.

### BACKGROUND OF THE INVENTION

The following includes information that may be useful in understanding the present disclosure. It is not an admission that any of the information provided herein is prior art nor material to the presently described or claimed inventions, nor that any publication or document that is specifically or implicitly referenced is prior art.

### 1. Field of the Invention

The present invention relates generally to the field of cosmetics and more specifically relates to a makeup storage and application system.

### 2. Description of Related Art

Cosmetics, also known as make-up, are substances or products used to enhance the appearance or fragrance of the body. Many cosmetics are designed for use of applying to the face and hair. In the 21st century, women generally use more cosmetics than men. They are generally mixtures of chemical compounds; some being derived from natural sources, and some being synthetics. Common cosmetics include lipstick, mascara, eye shadow, foundation, rouge, skin cleansers and skin lotions, shampoo, hairstyling products (gel, hair spray, etc.), perfume and cologne. Makeup can become expensive and acquiring just the right colors and combinations for a desired look may be difficult. A suitable solution is desired.

U.S. Pub. No. 2016/0206549 to Joseph Abernathy Lewis II, et al. relates to a botanical antioxidant compositions. The described botanical antioxidant compositions includes a botanical antioxidant composition having a botanical antioxidant extract blend with at least 50 wt. % of the blend having one or more hydroxycinnamic acid, and: a first component comprising a camellia sinesis extract, a vitis vinifera extract, a euterpe oleracea extract, a curcuma longa extract, or a theobroma cacao extract; and a second component, different from the first component, comprising the camellia sinesis extract, the vitis vinifera extract, the euterpe oleracea extract, the curcuma longa extract, or the theobroma cacao extract; wherein the composition is a cosmetic or dermatological formulation in a cream, lotion, solution, serum, anhydrous preparation, emulsion, microemulsion, multiple emulsion, gel, solid stick, ointment, dry powder, spray, or aerosol form.

### BRIEF SUMMARY OF THE INVENTION

In view of the foregoing disadvantages inherent in the known cosmetics art, the present disclosure provides a novel makeup storage and application system. The general purpose of the present disclosure, which will be described subsequently in greater detail, is to provide a custom makeup experience allowing a user to create custom combinations of makeup.

A makeup storage and application system is disclosed herein. The makeup storage and application system may include a makeup storage and application assembly having a makeup palette for holding a base powder, a wax, a cream, and at least one pigment. The makeup palette may be configured to hold the base powder, the wax, the cream and at least one pigment. At least one pigment is able to be interchangeably mixed from the makeup palette with the base powder, the wax, and the cream to create a customized makeup for the user.

The makeup palette may comprise a three-quarter cut away rotatable disc. The makeup palette may further comprise at least one pigment-bowl on a top surface. The three-quarter rotatable disc may be rotated about a center of the makeup palette exposing at least one of the pigment-bowls.

The makeup palette may also include a center compartment which may be used to house the base powder, the wax, the cream. At least one pigment may be interchangeably mixed with the base powder, the wax, and the cream to create a customized makeup for a user. The makeup palette may further include magnets located under a base portion of the makeup palette. The at least one pigment-bowl may be configured to be removable and refillable to allow for customization and extended use of the makeup palette.

A kit for a makeup storage and application system is also disclosed herein including a makeup palette, an application and mixing brush, and a set of user instructions.

The method of using makeup storage and application system may comprise the steps of: choosing a base, choosing at least one pigment, mixing said base with said at least of pigment to create a customized makeup from a makeup palette comprising a base powder, a wax, a cream, and at least one pigment, and applying of said customized makeup to a face of a user.

For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and methods of use for the present disclosure, a makeup storage and application system, constructed and operative according to the teachings of the present disclosure.

FIG. 1 is a front perspective view of the makeup storage and application system during an ‘in-use’ condition, according to an embodiment of the disclosure.

FIG. 2 is a front perspective view of the makeup storage and application system of FIG. 1 having a makeup palette, according to an embodiment of the present disclosure.

FIG. 3 is a front perspective view of the makeup palette of FIG. 1, according to an embodiment of the present disclosure.

FIG. 4 is a kit of the makeup storage and application system of FIG. 1, according to an embodiment of the present disclosure.

FIG. 5 is a flow diagram illustrating a method of use for the makeup storage and application system, according to an embodiment of the present disclosure.

The various embodiments of the present invention will hereinafter be described in conjunction with the appended drawings, wherein like designations denote like elements.

### DETAILED DESCRIPTION

As discussed above, embodiments of the present disclosure relate to cosmetics and more particularly to a makeup storage and application system as used to improve the user customization of makeup.

Generally, the present invention includes textures consisting of cream, powder and wax. The cream serves as a base to prepare skin for makeup and gives a creaseless setting for future powder application. The cream moisturizes skin and helps makeup stay on longer. The cream base comprises key ingredients including neem leaf oil, wheatgrass germ oil and sea of buckthorn extract. Olive oil infused with calendula, comfrey and chamomile may also be added to help calm irritated skin. Finally, a seaweed extract may be added for color correction to redness in skin. The cream base may be mixed with pigments for cream eye shadows, lip colors, blush, concealer, foundation and highlighter.

The wax may serve as a texture for lip product and eyebrow tint. The main active ingredients are organic beeswax, jojoba oil, rosehip oil, and calendula extract. The wax may be used to create custom makeup for a user.

The powder base may include a translucent base containing bamboo silica, diamond and pearl extract powders to give skin a glow. When mixed with a pigment the powder base may be used as a facial powder, bronzer, eyeshadow or blush. The makeup palette may offer 8 pigments per palette allowing a user to mix and create customized makeup including cream blush, highlighter, bronzer, eye shadow, and lip tint.

The powdered pigments contain botanical extracts. The pigments may include black, brown, pink, red, yellow, beige, green, and tan. Other pigments may be included based on user preference and manufacturing. Each palette collection may feature a different range in colors. The powder pigments may come in loose powder form fully sealed in tin containers. The key ingredients in the botanical extracts are watermelon, grapeseed, sandalwood and green tea extracts. Botanical extracts may be used because they include anti-oxidants and have anti-aging properties.

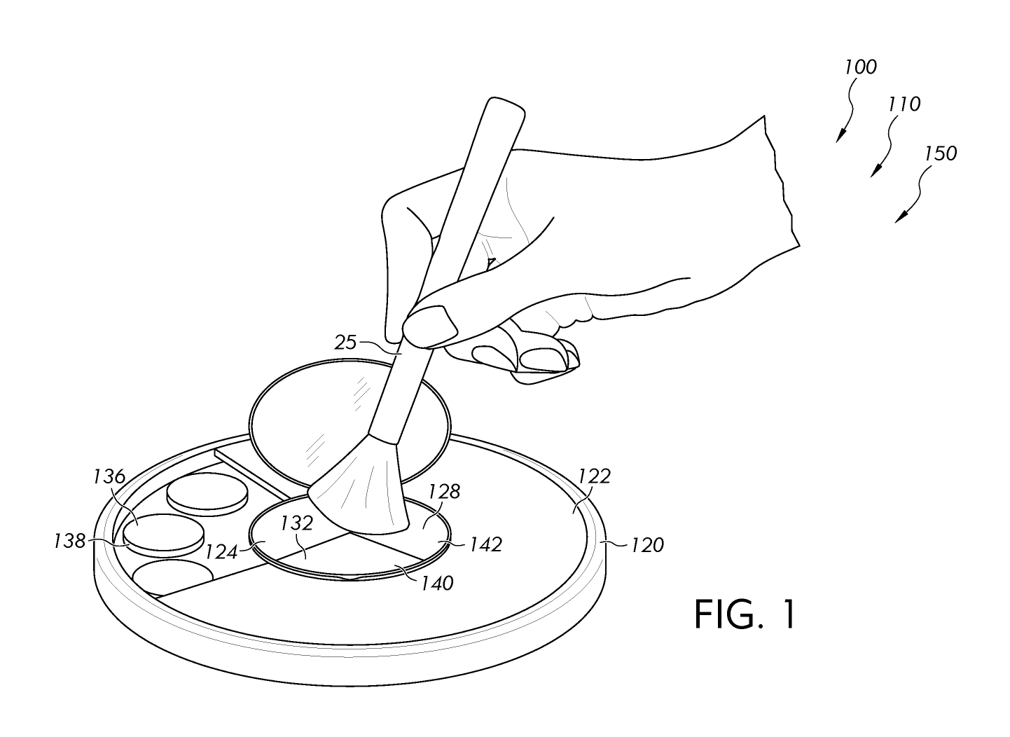

Referring now more specifically to the drawings by numerals of reference, there is shown in FIGS. 1-5, various views of a makeup storage and application system 100. FIG. 1 shows a makeup storage and application system 100 during an ‘in-use’ condition 150, according to an embodiment of the present disclosure. As illustrated, the makeup storage and application system 100 may include a makeup storage and application assembly 110 having a makeup palette 120 for holding a base powder 124, a wax 128, a cream 132, and at least one pigment 136. The makeup palette 120 is configured to hold the base powder, 124 the wax 128, the cream 132 and the at least one pigment 136. The at least one pigment 136 is able to be interchangeably mixed from the makeup palette 120 with the base powder 124, the wax 128, and the cream 132 to create a customized makeup for a user.

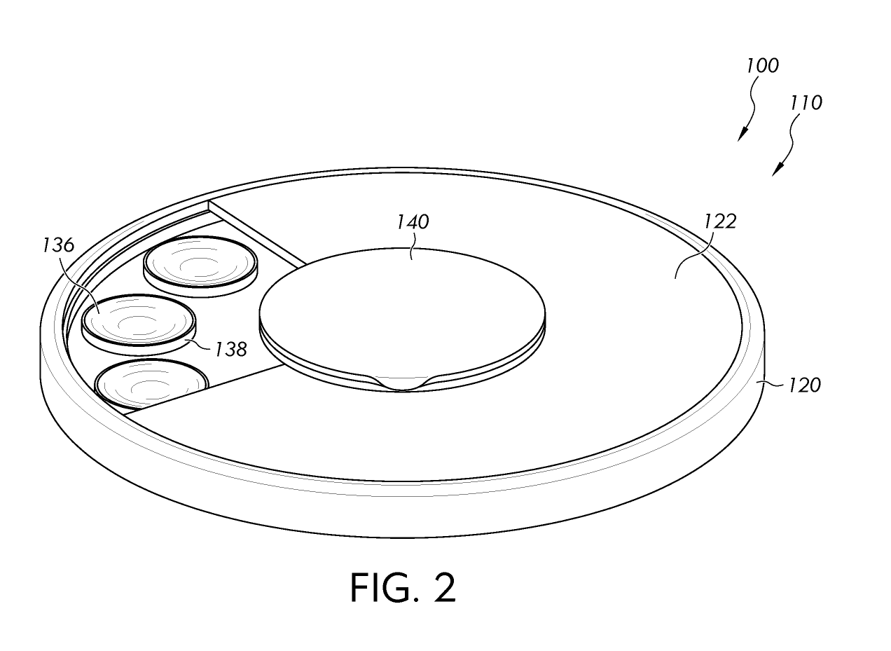

FIG. 2 shows a front perspective view of the makeup storage and application system 100 of FIG. 1, according to an embodiment of the present disclosure. As above, the makeup storage and application system 100 may include a makeup palette 120 comprising a three-quarter cut away rotatable disc 122. The three-quarter cut away rotatable disc 122 may rotate about a center of the makeup palette 120 as per user-manipulation. The makeup palette 120 may comprise at least one pigment-bowl 138 mounted to a top surface of the makeup palette 120. The at least one pigment-bowl 138 may hold a plurality of pigments 136 for user customization.

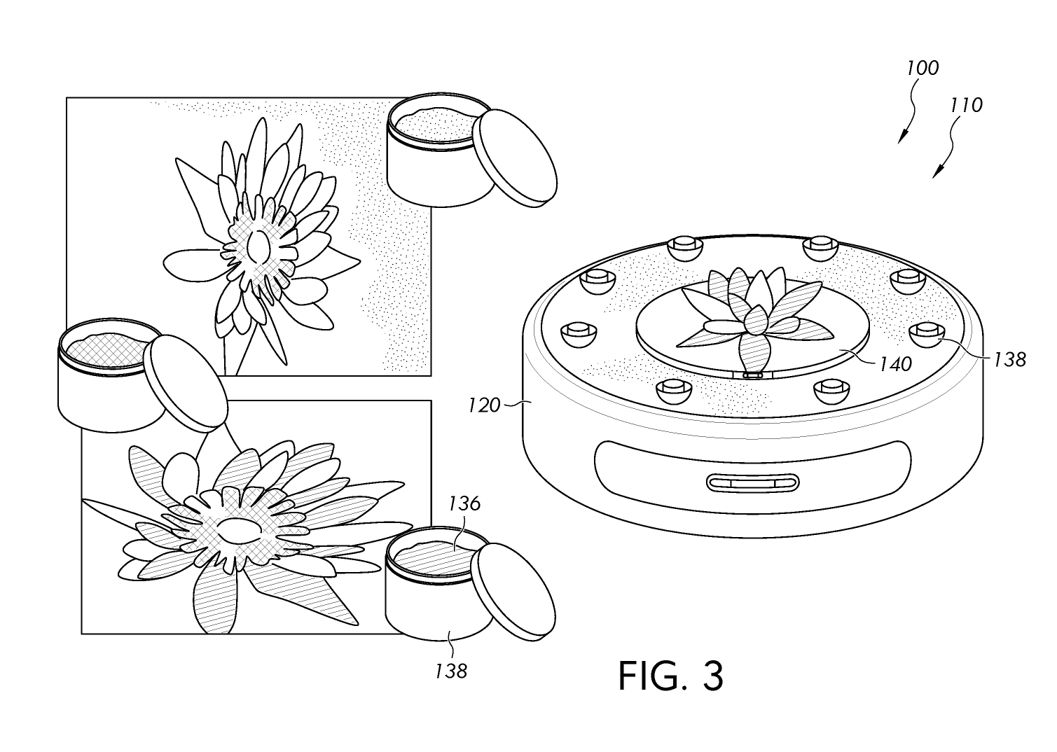

FIG. 3 shows a front perspective view of the makeup storage and application system 100 of FIG. 1, according to an embodiment of the present disclosure. As above, the makeup storage and application system 100 may include a makeup storage and application assembly 110 having a makeup palette 120 with a three-quarter cut away rotatable disc 122 which may rotate about a center of the makeup palette 120 as per user-manipulation.

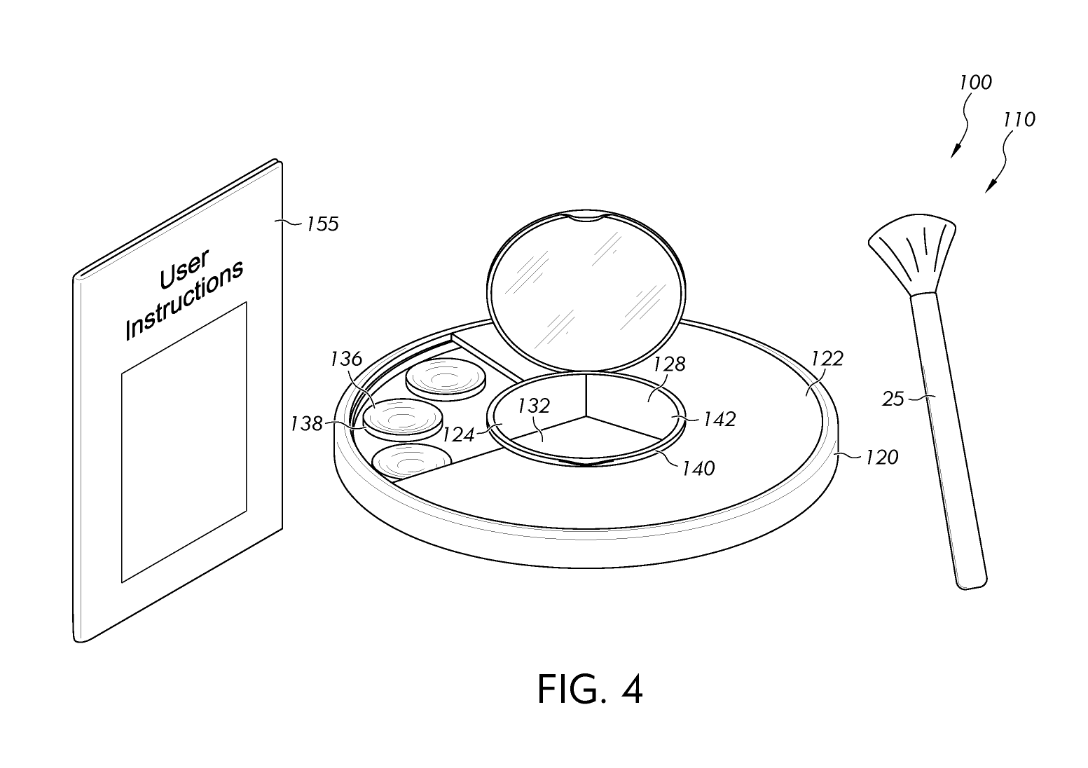

FIG. 4 shows a front perspective view of the makeup storage and application system 100 of FIG. 1, according to an embodiment of the present disclosure. As above, the makeup storage and application system 100 may include a center compartment 140 configured to house the base powder 124, the wax 128, the cream 132, and at least one pigment 136 such that the at least one pigment 136 is able to be interchangeably mixed with the base powder 124, the wax 128, the cream 132 to create a customized makeup for the user. At least one pigment-bowl 138 may be configured to be removable and refillable. The makeup palette 120 may further comprise magnets located in a base portion. The magnets may be useful for opening and closing the center compartment 1

In one embodiment of the present disclosure, a first phase of the base powder 124 may comprise Oryza Sativa (Rice) Starch 29.0%, Bismuth Oxychloride 25.0%, Boron Nitride 22.0%, Kaolinite (White Clay) 3.0%, Phyllostachys Glauca McClure (Bamboo Leaf) Botanical Extract 4.0%, Powder Silk 3.0%, Titanium Dioxide 1.0%, and Diamond Extract PF 1.0%; a second phase of the base powder 124 may comprise Simmondsia chinensis (Jojoba) Oil 4.25%, Camellia Oleifera (Camellia) Seed Oil 2.0%, Parfum 4.0%, Dimethicone 2.0%, and Preservatives 0.75%.

In one embodiment of the present disclosure, a first phase of the wax 128 may comprise Cocos nucifera (Coconut) Oil 29.5%, Cera Alba (Beeswax) 22.5%, Prunus dulcis (Sweet Almond) Oil 18.0%, Camellia oleifera (Camellia) Seed Oil 10.25%, Simmondsia chinensis (Jojoba) Oil 8.25%, Goosefat 4.0%, Polawax™ NF (Cetearyl Alcohol, PEG-150 Stearate, Polysorbate 60, and Steareth-20) 3.0%; a second phase of the wax 128 may comprise Fragrance 2.0%, Pterocarpus santalinus Powder 1.0%, Tocopherol 0.75%, and Optiphen Plus 0.75%.

In one embodiment of the present disclosure, a first phase of the cream 132 may comprise Olea Europaea oil 8.0%, Goose Fat/Graisse d'oie 2.0%, Triticum Vulgare Oil 3.0%, Polawax™ NF 4.0%, Rosa Canina Fruit Oil 3.0%, Dimethicone 3.0%, Camellia Oleifera Seed Oil 3.0%, Cera Alba 4.5%, and Azadirachta indica Leaf Oil 2.0%; a second phase of the cream 132 may comprise Aqua (Water) 50%, Glycerin 3.25%, Hippophae Rhamnoides Botanical Extract 1.5%, Calendula Officinalis Botanical Extract 1.5%, Matricaria Recutita Botanical Extract 1.5%, Symphytum Officinale Powder Extract 1.5%, and Ascophyllum Nodosum Powder Extract 1.0%; a third phase of the cream 132 may comprise Zinc 2.0%; a fourth phase of the cream 132 may comprise Parfum 3.5%, and Optiphen Plus (Phenoxyethanol, Caprylyl Glycol, Sorbic Acid) 1.75%.

At least one the pigment 136 may comprise black pigmentation. A first phase of the black at least one pigment 136 may comprise Oryza Sativa (Rice) Starch 28.0%, Bismuth Oxychloride 24.0%, Boron Nitride 20.0%, Activated Charocle 5.0%, Kaolin 3.0%, Vaccinium corymbosum Fruit Extract 4.0%, and Lavandula angustifolia Flower Extract 3.0%. A second phase of the black at least one pigment 136 may comprise Simmondsia chinensis (Jojoba) Oil 4.25%, Camellia Oleifera Seed Oil 2.0%, Parfum 4.0%, Dimethicone 2.0%, and Preservatives 0.75%.

The at least one pigment 136 may further comprise a yellow appearance. A first phase of the yellow at least one pigment 136 may comprise Oryza Sativa Starch 29.0%, Bismuth Oxychloride 25.0%, Boron Nitride 22.0%, Bamboo Leaf Botanical Extract 3.0%, Kaolin 2.0%, Ananus Communis Fruit Extract 3.0%, and Carica Papaya Fruit Extract 3.0%. A second phase of the yellow at least one pigment 136 may comprise Simmondsia chinensis (Jojoba) Oil 4.25%, Camellia Oleifera (Camellia) Seed Oil 2.0%, Parfum 4.0%, Dimethicone 2.0%, and Preservatives 0.75%;

At least one pigment 136 may include a bronze brown appearance. A first phase of the bronze brown at least one pigment 136 may comprise Oryza Sativa (Rice) Starch 29%, Bismuth Oxychloride 25.0%, Boron Nitride 22.0%, Panax ginseng meyer (Ginseng Root) Botanical Extract 3.0%, Pterocarpus santalinus (Sandalwood) Powder 3.0%, Kaolin 2.0%, Aspen Bark 3.0%. A second phase of the bronze brown at least one pigment 136 may comprise Simmondsia chinensis (Jojoba) Oil 4.25%, Camellia Oleifera (Camellia) Seed Oil 2.0%, Parfum 4.0%, Dimethicone 2.0%, and Preservatives 0.75%;

At least one pigment 136 may comprise a rose colored appearance. A first phase of the rose at least one pigment 136 may comprise Oryza Sativa (Rice) Starch 30%, Bismuth Oxychloride 25.0%, Boron Nitride 22.0%, Bamboo Leaf (70% Silica) Botanical Extract 3.0%, Fragaria Vesca (Strawberry) Fruit Extract 2.0%, Kaolin 2.0%, Citrullus Lanatus (Watermelon) Fruit Extract 3.0%. A second phase of the rose at least one pigment 136 may comprise Simmondsia chinensis (Jojoba) Oil 4.25%, Camellia Oleifera (Camellia) Seed Oil 2.0%, Parfum 4.0%, Dimethicone 2.0%, and Preservatives 0.75%.

At least one pigment 136 may include a rouge appearance. A first phase of the rouge at least one pigment 136 may comprise Oryza Sativa (Rice) Starch 30%, Bismuth Oxychloride 25.0%, Boron Nitride 22.0%, Rubia Tinctorum (Maddar) Root Powder 3.0%, Carmine (Ci 75470) 2.0%, Kaolin 2.0%, Rosa Canina (Rosehips) Botanical Extract 2.0%. A second phase of the rouge at least one pigment 136 may comprise Simmondsia chinensis (Jojoba) Oil 4.25%, Camellia Oleifera (Camellia) Seed Oil 2.0%, Parfum 4.0%, Dimethicone 2.0%, and Preservatives 0.75%.

The makeup palette 120 may be configured to hold the base powder 124, the wax 128, the cream 132 and the at least one pigment 136, the at least one pigment 136 having the following ingredients:

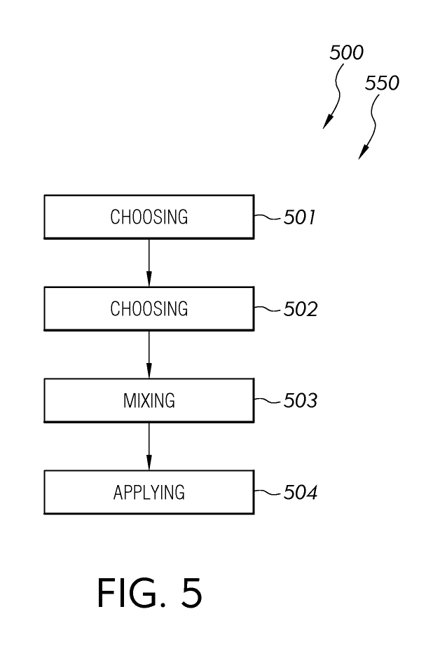

FIG. 5 is a flow diagram 550 illustrating a makeup storage and application system 500, according to an embodiment of the present disclosure. As illustrated, the makeup storage and application system 500 may include the steps of: step one 501, choosing a base power 124, step two 502, choosing at least one pigment 136, step three 503, mixing the base 124 with at least of pigment 136 to create a customized makeup from a makeup palette 120, and step four 504, applying of the customized makeup to a face of a user.

It should also be noted that the steps described in the method of use can be carried out in many different orders according to user preference. The use of “step of” should not be interpreted as “step for”, in the claims herein and is not intended to invoke the provisions of 35 U.S.C. § 112(f). It should also be noted that, under appropriate circumstances, considering such issues as design preference, user preferences, marketing preferences, cost, structural requirements, available materials, technological advances, etc., other methods for makeup storage and application system 100 (e.g., different step orders within above-mentioned list, elimination or addition of certain steps, including or excluding certain maintenance steps, etc.), are taught herein.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is new and desired to be protected by Letters Patent is set forth in the appended claims:

1. A makeup storage and application system comprising:
a makeup storage and application assembly including;
a makeup palette for holding;
a base powder;
a wax;
a cream; and
at least one pigment;
wherein said makeup palette is configured to hold said base powder, said wax, said cream and said at least one pigment, said at least one pigment is able to be interchangeably mixed from said makeup palette with said base powder, said wax, and said cream to create a customized makeup for a user.

2. The makeup storage and application system of claim 1, wherein said makeup palette comprises a three-quarter cut away rotatable disc.

3. The makeup storage and application system of claim 2, wherein said three-quarter cut away rotatable disc rotates about a center of said makeup palette as per user-manipulation.

4. The makeup storage and application system of claim 3, wherein said makeup palette comprises at least one pigment-bowl mounted to a top surface of said makeup palette.

5. The makeup storage and application system of claim 4, wherein said makeup palette includes a center compartment.

6. The makeup storage and application system of claim 5, wherein said center compartment is configured to house said base powder, said wax, said cream, and at least one pigment such that said at least one pigment is able to be interchangeably mixed with said base powder, said wax, and said cream to create a customized makeup for said user.

7. The makeup storage and application system of claim 4, wherein said at least one pigment-bowl is configured to be removable and refillable.

8. The makeup storage and application system of claim 1, wherein said makeup palette comprises magnets located in a base portion.

9. The makeup storage and application system of claim 1, wherein a first phase of said base powder comprises Oryza Sativa Starch 29.0%, Bismuth Oxychloride 25.0%, Boron Nitride 22.0%, Kaolinite 3.0%, Phyllostachys Glauca McClure Botanical Extract 4.0%, Powder Silk 3.0%, Titanium Dioxide 1.0%, and Diamond Extract PF 1.0%.

10. The makeup storage and application system of claim 9, wherein a second phase of said base powder comprises Simmondsia chinensis Oil 4.25%, Camellia Oleifera Seed Oil 2.0%, Parfum 4.0%, Dimethicone 2.0%, and Preservatives 0.75%.

11. The makeup storage and application system of claim 1, wherein a first phase of said wax comprises Cocos nucifera Oil 29.5%, Cera Alba 22.5%, Prunus dulcis Oil 18.0%, Camellia oleifera Seed Oil 10.25%, Simmondsia chinensis Oil 8.25%, and Goosefat 4.0%, Polawax™ NF (Cetearyl Alcohol, PEG-150 Stearate, Polysorbate 60, and Steareth-20) 3.0%.

12. The makeup storage and application system of claim 11, wherein a second phase of said wax comprises Fragrance 2.0%, Pterocarpus santalinus Powder 1.0%, Tocopherol 0.75%, and Optiphen Plus 0.75%.

13. The makeup storage and application system of claim 1, wherein a first phase of said cream comprises Olea Europaea oil 8.0%, Goose Fat/Graisse d'oie 2.0%, Triticum Vulgare Oil 3.0%, Polawax™ NF 4.0%, Rosa Canina Fruit Oil 3.0%, Dimethicone 3.0%, Camellia Oleifera Seed Oil 3.0%, Cera Alba 4.5%, and Azadirachta indica Leaf Oil 2.0%.

14. The makeup storage and application system of claim 13, wherein a second phase of said cream comprises Aqua 50%, Glycerin 3.25%, Hippophae Rhamnoides Botanical Extract 1.5%, Calendula Officinalis Botanical Extract 1.5%, Matricaria Recutita Botanical Extract 1.5%, Symphytum Officinale Powder Extract 1.5%, and Ascophyllum Nodosum Powder Extract 1.0%.

15. The makeup storage and application system of claim 14, wherein a third phase of said cream comprises Zinc 2.0%.

16. The makeup storage and application system of claim 15, wherein a fourth phase of said cream comprises Parfum 3.5%, Optiphen Plus (Phenoxyethanol, Caprylyl Glycol, Sorbic Acid) 1.75%.

17. The makeup storage and application system of claim 1, wherein said at least one pigment is able to be mixed with said base powder, said wax, and said cream to create a customized makeup for said user.

18. A makeup storage and application system comprising:
a makeup storage and application assembly including;
a makeup palette for holding;
a base powder;
a wax;
a cream; and
at least one pigment;
wherein said makeup palette comprises a three-quarter cut away rotatable disc;
wherein said makeup palette comprises at least one pigment-bowl on a top surface;
wherein said three-quarter rotatable disc rotates about a center of said makeup palette exposing at least one of said pigment-bowls;
wherein said makeup palette includes a center compartment;
wherein said center compartment housing of said makeup palette comprising said base powder, said wax, said cream, and at least one pigment said at least one pigment is able to be interchangeably mixed with said base powder, said wax, and said cream to create a customized makeup for a user;
wherein said makeup palette comprises magnets located under a base portion of said makeup palette;
wherein said at least one pigment-bowl is configured to be removable and refillable;
wherein a first phase of said base powder comprises Oryza Sativa Starch 29.0%, Bismuth Oxychloride 25.0%, Boron Nitride 22.0%, Kaolinite 3.0%, Phyllostachys Glauca McClure Botanical Extract 4.0%, Powder Silk 3.0%, Titanium Dioxide 1.0%, and Diamond Extract PF 1.0%;
wherein a first phase of said wax comprises Cocos nucifera Oil 29.5%, Cera Alba 22.5%, Prunus dulcis Oil 18.0%, Camellia oleifera Seed Oil 10.25%, Simmondsia chinensis Oil 8.25%, and Goosefat 4.0%, Polawax™ NF (Cetearyl Alcohol, PEG-150 Stearate, Polysorbate 60, and Steareth-20) 3.0%;
wherein a second phase of said wax comprises Fragrance 2.0%, Pterocarpus santalinus Powder 1.0%, Tocopherol 0.75%, and Optiphen Plus 0.75%;
wherein a first phase of said cream comprises Olea Europaea oil 8.0%, Goose Fat/Graisse d'oie 2.0%, Triticum Vulgare Oil 3.0%, Polawax™ NF 4.0%, Rosa Canina Fruit Oil 3.0%, Dimethicone 3.0%, Camellia Oleifera Seed Oil 3.0%, Cera Alba 4.5%, and Azadirachta indica Leaf Oil 2.0%;
wherein a second phase of said cream comprises Aqua 50%, Glycerin 3.25%, Hippophae Rhamnoides Botanical Extract 1.5%, Calendula Officinalis Botanical Extract 1.5%, Matricaria Recutita Botanical Extract 1.5%, Symphytum Officinale Powder Extract 1.5%, and Ascophyllum Nodosum Powder Extract 1.0%;
wherein a second phase of said base powder comprises Simmondsia chinensis Oil 4.25%, Camellia Oleifera Seed Oil 2.0%, Parfum 4.0%, Dimethicone 2.0%, and Preservatives 0.75%;
wherein a third phase of said cream comprises Zinc 2.0%;
wherein a fourth phase of said cream comprises Parfum 3.5%, Optiphen Plus (Phenoxyethanol, Caprylyl Glycol, Sorbic Acid) 1.75%; and
wherein said makeup palette is configured to hold said base powder, said wax, said cream and said at least one pigment, said at least one pigment is able to be interchangeably mixed with said base powder, said wax, and said cream to create a customized makeup for said user.

19. The makeup storage and application system of claim 18, further comprising a kit comprising:
said makeup palette;
an application and mixing brush; and
a set of user instructions.

20. A method of using a makeup storage and application system comprising the steps:
choosing a base;
choosing at least one pigment;
mixing said base with said at least of pigment to create a customized makeup from a makeup palette comprising a base powder, a wax, a cream, and said at least one pigment; and
applying of said customized makeup to a face of a user.
